(19) United States
(12) Patent Application Publication
(10) Pub. No.: US 2021/0008303 A1
(43) Pub. Date: Jan. 14, 2021

(54) VAPOR MIXING APPARATUS

(71) Applicant: Cannetic, Inc., San Jose, CA (US)

(72) Inventor: Darran Bahl, San Jose, CA (US)

(21) Appl. No.: 16/509,489

(22) Filed: Jul. 11, 2019

(51) Int. Cl. A61M 15/00; A24F 47/00; A61M 11/04

(52) U.S. Cl. CPC A61M 15/0003; A24F 47/008; F17C 9/02; A61M 15/0086; A61M 15/002; A61M 11/042

(57) ABSTRACT

A vapor blending device is provided, generally having a base, a carrier removably couplable to the base for carrying a first and second vapor generating system. The first and second vapor generating systems including airflow generators and controllers configured to vary voltage to each of the first and second vapor generating devices and the airflow generators. A container is positioned in fluid communication with the first and second vapor generating systems and configured to retain a quantity of vapor. In some examples, one controller is configured to cause the first vapor generating system to generate a quantity of a first vapor, and another controller is configured to cause the second vapor generating system to generate a quantity of a second vapor in a predetermined ratio to the quantity of the first vapor. The vapor in the predetermined ratio is stored in the container for later removal.

### BACKGROUND

Non-combustion vapor inhalation devices have become increasingly popular for recreational and therapeutic use. Such inhalation devices generate an inhalable vapor by atomizing a solid or liquid solution with a heating element. The atomized solution provides a delivery mechanism for introducing substances and chemicals into the human body for bloodstream absorption through the lungs. In some examples, conventional inhalation devices are used to atomize mixtures of propylene glycol, polyethylene glycol, and/or glycerin, among others, combined with chemicals such as nicotine, cannabidiol (CBD), tetrahydrocannabidnol (THC), etc. In other examples, conventional inhalation devices are used to directly atomize a solid plant-based or chemical substance, such as tobacco, cannabis, medications, etc., and combinations thereof.

Generally, non-combustion vapor inhalation devices provide a more efficient delivery of the atomized substance as combustion methods typically cause thermal decomposition, especially in organic materials. In this regard, a greater concentration of the substance can be delivered to the human body through non-combustion inhalation. Some users prefer to prepare a quantity of vapor for storage during inhalation sessions. For example, vapor may be generated by the non-combustion inhalation device and stored in an airtight container, such as a plastic bag, and then smaller doses of the vapor can be removed during the inhalation session.

One type of vapor-generating inhalation device is an elongate tube, commonly referred to as an electronic cigarette, e-cigarette, e-cig, vaporizer pen, vape pen, or the like, for resembling the shape and size of conventional cigarettes, cigars, pens, etc. Such devices are popular due to their compact form factor, ergonomic shape, and general availability of various cartridges containing a wide variety of solutions to align with user need and/or preference. Although a wide variety of atomizable solids and solutions exist for use with inhalation devices, a single cartridge is generally used with each electronic cigarette during inhalation. Greater flexibility is desirable to cater to the taste of a user. Therefore, a need exists for portable additional mixing flexibility such that the solid or liquid solution can be tailored to the physiological system and personal preference of a user.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with one embodiment of the present disclosure, a vapor blending device is provided. The vapor blending device generally includes a base, a carrier removably couplable to the base and having a first aperture and a second aperture, a first vapor generating system, and a second vapor generating system. The first vapor generating system generally includes a first vapor generating device positioned within the first aperture, a first airflow generator in fluid communication with the first vapor generating device, and a first controller in electrical communication with the first vapor generating device and the first airflow generator, the first controller configured to vary voltage to one or more of the first vapor generating device and the first airflow generator. The second vapor generating system generally includes a second vapor generating device positioned within the second aperture, a second airflow generator in fluid communication with the second vapor generating device, and a second controller in electrical communication with the second vapor generating device and the second airflow generator, the second controller configured to vary voltage to one or more of the second vapor generating device and the second airflow generator. The vapor blending device further includes a container in fluid communication with the first and second vapor generating systems and configured to retain a quantity of vapor, wherein the first controller may be configured to cause the first vapor generating system to generate a quantity of a first vapor, and wherein the second controller may be configured to cause the second vapor generating system to generate a quantity of a second vapor in a predetermined ratio to the quantity of the first vapor.

In accordance with another embodiment of the present disclosure, a vapor blending device is provided. The vapor blending device generally includes a carrier having a first aperture and a second aperture, a first vapor generating device positioned within the first aperture and in fluid communication with a first airflow generator having a first controller in electrical communication with the first vapor generating device and the first airflow generator, the first controller configured to vary voltage to one or more of the first vapor generating device and the first airflow generator, a second vapor generating device positioned within the second aperture and in fluid communication with a second airflow generator having a second controller in electrical communication with the second vapor generating device and the second airflow generator, the second controller configured to vary voltage to one or more of the second vapor generating device and the second airflow generator, and a container in fluid communication with the first and second vapor generating devices and configured to retain a quantity of vapor, wherein the first controller may be configured to cause the first vapor generating system to generate a quantity of a first vapor, and wherein the second controller may be configured to cause the second vapor generating system to generate a quantity of a second vapor.

In any of the embodiments described herein, the vapor generating device may further include a third vapor generating system generally including a third vapor generating device positioned within a third aperture of the carrier, a third airflow generator in fluid communication with the third vapor generating device, and a third controller in electrical communication with the third vapor generating device and the third airflow generator, the third controller configured to vary voltage to one or more of the third vapor generating device and the third airflow generator, wherein the third controller may be configured to cause the third vapor generating system to generate a quantity of a third vapor in a predetermined ratio to the quantity of the first and second vapors.

In any of the embodiments described herein, the vapor generating device may further include a fourth vapor generating system generally including a fourth vapor generating device positioned within a fourth aperture of the carrier, a fourth airflow generator in fluid communication with the fourth vapor generating device, and a fourth controller in electrical communication with the fourth vapor generating device and the fourth airflow generator, the fourth controller configured to vary voltage to one or more of the fourth vapor generating device and the fourth airflow generator, wherein the fourth controller may be configured to cause the fourth vapor generating system to generate a quantity of a fourth vapor in a predetermined ratio to the quantity of the first, second, and third vapors.

In any of the embodiments described herein, the vapor generating device may further include a receptacle configured to sealingly couple with the container, a sealing plate positioned between the receptacle and the carrier, the sealing plate having a selectively sealing aperture configured to seal the vapor in the container when the receptacle is removed from the carrier, and/or a sealing member positioned between the receptacle and the carrier, the sealing plate configured to prevent vapor from entering the base.

### DESCRIPTION OF THE DRAWINGS

The foregoing aspects and many of the attendant advantages of the present disclosure will become more readily appreciated as the same become better understood by reference to the following detailed description, when taken in conjunction with the accompanying drawings, wherein:

FIG. 1 is perspective view of a representative embodiment of a vapor blending device according to various aspects of the present disclosure;

FIG. 2 is a partially exploded perspective view of the vapor blending device of FIG. 1;

FIG. 3 is an exploded perspective view of the vapor blending device of FIG. 1; and

FIG. 4 is a top view of a representative embodiment of a carrier in accordance with another aspect of the present disclosure.

### DETAILED DESCRIPTION

The detailed description set forth below in connection with the appended drawings, where like numerals reference like elements, are intended as a description of various embodiments of the present disclosure and are not intended to represent the only embodiments. Each embodiment described in this disclosure is provided merely as an example or illustration and should not be construed as precluding other embodiments. The illustrative examples provided herein are not intended to be exhaustive or to limit the disclosure to the precise forms disclosed.

In the following description, specific details are set forth to provide a thorough understanding of exemplary embodiments of the present disclosure. It will be apparent to one skilled in the art, however, that the embodiments disclosed herein may be practiced without embodying all of the specific details. In some instances, well-known process steps have not been described in detail in order not to unnecessarily obscure various aspects of the present disclosure. Further, it will be appreciated that embodiments of the present disclosure may employ any combination of features described herein.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

The following description provides several examples that relate to configurations of apparatuses for generating, blending, and storing vapor from at least one inhalation device. In some embodiments of the present disclosure, the apparatuses provide a controlled mixture of vapor from two or more inhalation devices. In this regard, the vapor mixture may be stored in a container for metered and/or delayed inhalation. As described above, conventional inhalation devices are generally configured to atomize a solid or liquid solution using a heating element. The atomization creates a vapor that can be inhaled and introduced into the bloodstream of the user. In some embodiments, the inhalation device includes an integral cartridge for facilitating the interaction of the solid or solution for atomization with the heating element of the inhalation device.

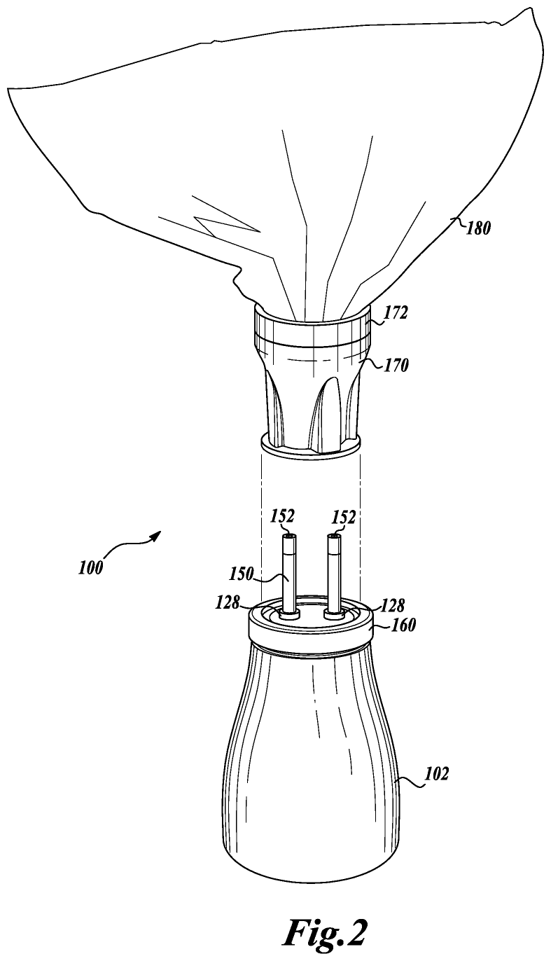

Such integral cartridges generally include a single chamber containing the solid or liquid solution. Inhalation through the exhaust aperture creates a low pressure vacuum through passageways in the inhalation device, such as an air intake aperture, and directs the air to carry the generated vapor to the exhaust aperture (see, e.g., FIG. 2, element 152). In some embodiments, the inhalation device senses the presence of a vacuum and activates the heating element to provide heat energy to the solution, causing atomization. As the user continues to inhale through the exhaust aperture, the vapor created from the solution travels to the lungs of the user. In these embodiments, the vapor generated by the inhalation device originates from the solution in the chamber of the integral cartridge. The integral cartridges typically contain a wide variety of mixtures of chemicals, compounds, solutions, and the like. In some examples, the integral cartridge contains a solution having certain doses of a chemical (e.g., a specified concentration of nicotine), various aromatics and flavors, and/or ratios of ingredients of a substance (e.g., the ratio of THC to CBD).

Although an inhalation device typically generates atomized solution in the form of vapor for immediate inhalation by the user, in some embodiments, the vapor is created to be captured and stored within a container for transfer, retrieval of a subset quantity of the vapor, and/or retrieval after a duration of time. In these storage embodiments, air pressure is generated to initiate positive airflow through the air intake and the passageways of the inhalation device to create the atomized vapor at the heating element and expel the vapor through the exhaust aperture. In embodiments where the vapor is created for storage, a container, especially an expandable container (e.g., a plastic bag), is positioned to create an airtight seal in proximity to the exhaust aperture of the inhalation device. Upon the generation of positive air pressure, the vapor travels into the container for storage.

Certain users of non-combustion inhalation devices have physiological and preference differences in regard to the doses, aromatics, and ratios of the vapor generated by the inhalation device. In this regard, a user-controlled mixture of multiple solutions provides greater flexibility to suit the preference of the user. In some embodiments described herein, a vapor mixing apparatus is provided to simultaneously generate vapor from two or more inhalation devices to create a mixture within the container. In these embodiments, the vapor mixing apparatus is configured to independently control the quantity of vapor generated by each inhalation device to create a user-specified mixture of vapor within the container. In certain embodiments, the vapor mixing apparatus includes a computer to control the mixture of vapor generated by the various inhalation devices.

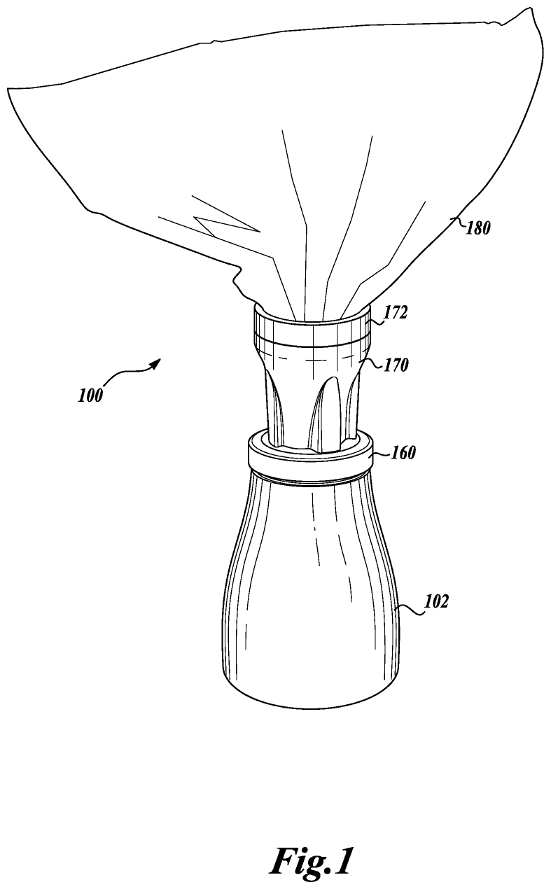

Referring initially to FIG. 1, one embodiment of a vapor blending device 100 is shown. The vapor blending device 100 generally includes a base 102, a coupler 160, a receptacle 170, a sealing member 172, and a container 180. In these embodiments, the base 102 is configured to provide a surface such that the vapor blending device 100 can be positioned on a counter, table, desk, etc. during use of the vapor blending device 100. The base 102 is additionally configured to mask certain components of the vapor blending device 100 for aesthetic and packaging purposes, including the air pressure devices and the power supply, which will be explained in greater detail below. In some embodiments, the base 102 includes a mechanical coupling feature, such as threads 104 to interface and couple the base 102 to other components of the vapor blending device 100. In other embodiments, the base 102 is suitably any shape, size, or configuration, or may be substantially omitted or integrated into other components, e.g., the base 102 may include a battery access door (not shown) to make an internal battery more accessible, a charging port, a data communication port, non-slip feet, lighting, a user display, etc. In some of these embodiments, the base 102 is configured such that the user cannot remove the base from the receptacle 170. In this regard, the coupler 160 may be omitted.

In the illustrated embodiment, the container 180 is a flexible bag coupled to the receptacle 170 and sealed by the sealing member 172 such that vapor generated by the vapor blending device 100 passes through the receptacle 170 and into the container 180 without leaking. In other embodiments, the container 180 may be sealed to the vapor blending device 100 using any conventional sealing method, such as by wrapping an open-end of the container 180 around a chamber within the receptacle 170 and creating a seal between the components, e.g., with an elastomeric band. In some embodiments, the container 180 is a flexible bag, such as those manufactured from high density polyethylene (HDPE), low-density polyethylene (LDPE), linear low-density polyethylene (LLDPE), medium density polyethylene (HDPE), polypropylene (PP), etc. Although the container 180 is illustrated as a flexible bag, in other embodiments, any suitable container is within the scope of the present disclosure.

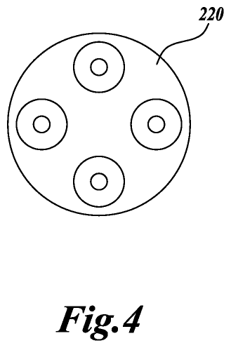

Turning now to FIG. 2, a partially exploded view of the vapor blending device 100 is shown, where the receptacle 170 is separated from the coupler 160 to expose several internal components of the vapor blending device 100. The illustrated embodiment of the vapor blending device 100 includes a plurality of vapor generating devices 150 (e.g., a cartridge, electronic cigarette, e-cigarette, e-cig, vaporizer pen, vape pen, etc.). Each vapor generating device 150 includes an exhaust aperture 152 through which the vapor exits the vapor generating device 150. For simplicity and the FIGURES and the ensuing description, the illustrations depict two vapor generating devices 150 for use with the vapor blending device 100. However, in other embodiments, the vapor blending device 100 is configured to interface and control any number of vapor generating devices 150, for example, an array of four vapor generating devices 150 (see FIG. 4). In this regard, the vapor blending device 100 can be expanded to interface and control a large number of vapor generating devices 150 such that the user can develop a personalized blend of vapor within the container 180 for inhalation. In some embodiments, it is advantageous to seal the interface between the vapor generating device 150 and coupler 160 such that the vapor generated during use of the vapor blending device 100 does not leak past the coupler 160 and into base 102.

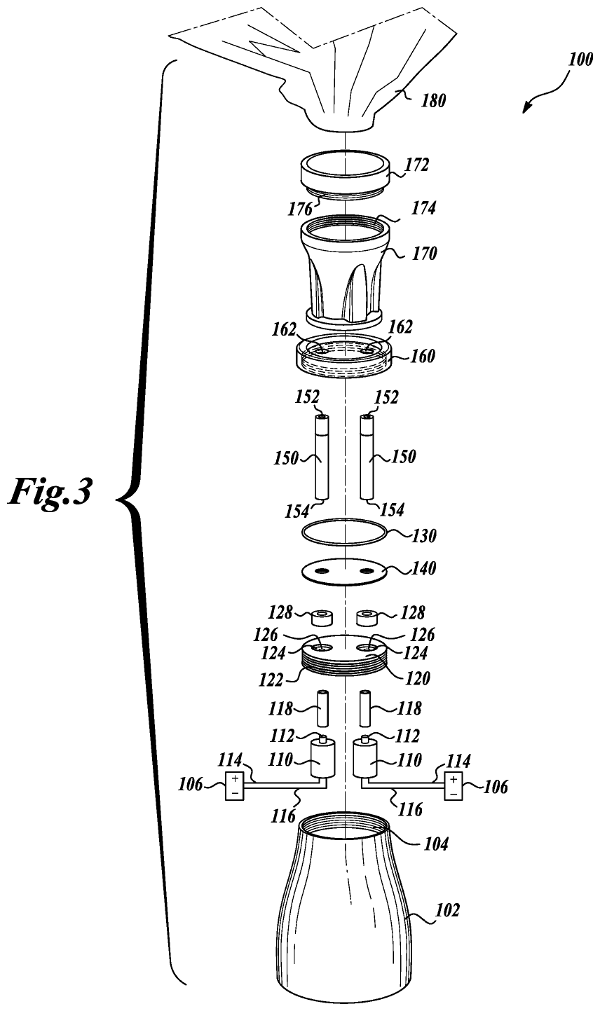

Referring to FIG. 3, there is shown an exploded view of the vapor blending device 100, exposing the internal components. Referring initially to the air delivery section of the vapor blending device 100, a blower 110 (or airflow generator) is positioned in fluid communication with each of the vapor generating devices 150. In some embodiments, the blower 110 is any suitable pressure generating airflow device, such as a pump, scroll fan, compressed air delivery, etc. As illustrated, in some embodiments, the blower 110 includes an air outlet 112 for delivery of pressurized air through a tube 118 to the vapor generating devices 150, ultimately forcing the vapor into the container 180. In some embodiments, the tube 118 is flexible such that movement or vibration from the blowers 110 is not directly transferred to the vapor generating devices 150. In certain embodiments, the blower 110 is an electrically controlled device, such as an electric motor connected to an air pressure generator. In this regard, the blower 110 may include a positive lead 114 and a negative lead 116, electrically coupling the blower 110 to a controller 106.

During use of the vapor blending device 100, the user may specify different characteristics of the blends of vapor from the vapor generating devices 150. In several embodiments, the vapor blending device 100 includes controls to provide differential air pressures traveling through the vapor generating devices 150, such as by varying the voltage applied by the controller 106 to the blowers 110. In these embodiments, to generate a specified blend of vapor, the pressure generated by each blower 110 may be different from each of the other blowers 110 to provide a mixture of vapor from various sources within the vapor blending device 100. In one example, a user may load the vapor blending device 100 with selected vapor generating devices 150, each including a different cartridge having the solid or liquid solution desired to form a portion of the final vapor combination in the container 180. In some embodiments, any suitable cartridge can be used with the vapor blending device 100. In one embodiment, a 510-threaded oil cartridge is used with the vapor blending device 100. In this embodiment, the 510-threaded oil cartridge may be positioned to interface directly with the tube 118, such that the cartridge is the vapor generating device 150, or the cartridge may be assembled to a housing to form the vapor generating device 150, such as an e-cigarette, and positioned within the vapor blending device 100 to interface with the tube 118 and other components.

In some embodiments, the vapor generating devices 150 are coupled to the vapor blending device 100 using a carrier 120 configured to interface the base 102. In these embodiments, the carrier 120 includes apertures 126 configured to receive the vapor generating devices 150. In embodiments where more than two apertures 126 are included in the carrier 120, the vapor blending device 100 may still be used with fewer vapor generating devices 100 than the number of apertures 126. In these embodiments, the unused aperture 126 may be sealed using a blank seal (not shown) such that the generated vapor does not leak past the unused aperture 126 into the base 102. In some embodiments, the carrier 120 includes threads 122 configured to interface the threads 104 of the base 102; however, in other embodiments, the carrier 120 includes any coupling feature to the base 102, such as twist to lock, press fit, magnets, constraint/stepped capture, etc.

As shown, the apertures 126 of the carrier 120 may include optional countersunk portions 124 such that a sealing washer 128 can be used to provide a seal between the vapor generating device 150 and the carrier 120. In addition to the sealing washer 128, a sealing plate 140 is configured to interface the upper surface of the carrier 120 to provide an additional seal between the carrier 120 and the vapor generating devices 150. In some embodiments, the sealing plate 140 includes mechanical sealing features, such as flaps (not shown) in the sealing plate 140. In these embodiments, as the receptacle 170 is coupled to the coupler 160, the flaps on the sealing plate 140 open airflow from the vapor generating devices 150 into the container 180. Then, when the receptacle 170 is disconnected from the coupler 160, the flaps on the sealing plate 140 substantially close and prevent airflow from the container 180. In other embodiments, any mechanical activating seal is within the scope of the present disclosure.

In some embodiments, a supplemental seal 130, such as an O-ring, is positioned between the sealing plate 140 and the coupler 160. In these embodiments, the supplemental seal 130 provides a seal between the mechanical connection of the carrier 120 and the coupler 160, such as by the threads 122 of the carrier 120 and the threads 162 of the coupler 160. In other embodiments, the coupler 160 and the base 102 mechanically capture the carrier 120 without using any positive mechanical coupling, such as magnetically. In these embodiments, the carrier 120 may include additional sealing members to provide a seal between the carrier 120, the coupler 160, and/or the base 102. In further embodiments, the carrier 120 may be integrated into the coupler 160 to form a single component, or may form any number or components.

Returning briefly to the receptacle 170, as shown in FIG. 3, in some embodiments, the receptacle 170 and the sealing member 172 may be joined using a mechanical coupling, such as by threads 176 of the sealing member 172 interfacing threads 174 of the receptacle 170. In other embodiments, any suitable coupling, such as magnetic, is within the scope of the present disclosure. In some embodiments, the opening of the container 180 is positioned between the sealing member 172 and the receptacle 170, such that the edge of the opening of the container 180 is clamped during the assembly of the sealing member 172 to the receptacle 170. In other embodiments, the container 180 is sealingly coupled to the receptacle 170 using any suitable technique. In some embodiments, the receptacle 170 releasably couples to the coupler 160 using any suitable technique, such as magnetically, twist to lock, press fit, constraint/stepped capture, etc. In one embodiment, the receptacle 170 may include additional threads at the base (not shown) and the coupler 160 may include corresponding additional threads (not shown). In other embodiments, the receptacle 170 couples to the coupler 160 with the press fit, which may include an additional sealing member such as an O-ring (not shown). In additional embodiments, when the container 180 is filled with vapor, the receptacle 170 may be removed from the remaining components of the vapor blending device 100 and a mouthpiece (not shown) may be connected to the housing such that a user can inhale the vapor. In this embodiment, the mouthpiece may function as a check valve such that vapor can only escape the container 180 when the user pulls a vacuum by inhaling around the mouthpiece opening. In these embodiments, the mouthpiece may be coupled to the receptacle 170 using a magnetic coupling, or any other suitable coupling.

Control of the vapor blending device 100 will now be described in greater detail. As shown in FIG. 3, in some embodiments, each blower 110 may be connected to a controller 106 having a power source (not shown). In some embodiments, the controller 106 is a microcontroller. The power source for the controller 106 is suitably any power source to cause the blower 110 to function. In some embodiments, the power source is portable, such as by using one or more batteries contained within the base 102. In other embodiments, the power source may be an alternating current source from a wall outlet.

In some embodiments, the controller 106 is configured to communicate with a user interface positioned on the vapor blending device 100, a remote control unit (e.g., a smartphone or computer running a control app communicating through BLUETOOTH® or Wi-Fi), and/or other controllers 106 within the vapor blending device 100, such as buttons, switches, dials, potentiometers, etc. Based on user input, computer-generated input, readings from sensors, recipes, feedback, stored data, etc. and/or combinations thereof, the controller 106 is configured to generate an amount of airflow through the vapor generating device 150 to cause the desired ratio of the vapor from the connected vapor generating device 150 to be transferred to and stored in the container 180. In addition, in certain embodiments, the controller 106 is configured to adjust the voltage, and thereby the temperature, of a heating element in the vapor generating device 150, such that the vapor quantity and density can be controlled.

In some embodiments, the vapor blending device 100 is configured to input data from the user, other users, the Internet, etc. to create specific blends of vapor in the container 180. Non-limiting examples of use of the vapor blending device 100 will now be described in greater detail. In one example, the user attaches up to four vapor generating devices 150 to the base 102 and couples the receptacle 170. The user then sets a desired vapor quantity from each vapor generating device 150 using the software interface. Once the user initiates the fill of the container 180, the software sends the instructions to the controllers 106 which operate the blowers 110 and heating elements of the vapor generating devices 150 to produce the desired vapor mixture in the container 180. In another example, the user can explore community/recommended recipes, based on experience, effect, etc. and/or the cartridges available to the user. In this regard, the software provides instructions for the user to place the correct cartridge in each aperture 126 and initiates the fill of the container 180.

Turning finally to FIG. 4, as described above, the vapor blending device 100 is suitable for use with any number of vapor generating devices 150. In the illustrated embodiment of FIG. 4, a carrier 220 includes four apertures 226, each optionally having a countersunk portion 224. As noted above, the carrier of the vapor blending device 100 may be modified to interface any number of vapor generating devices 150.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

1. A vapor blending device, comprising:
a base;
a carrier removably couplable to the base and having a first aperture and a second aperture;
a first vapor generating system comprising:
a first vapor generating device having a first heating element positioned within the first aperture, the first vapor generating device having a portion protruding through the carrier away from the base;
a first airflow generator positioned in the base and in fluid communication with the first vapor generating device; and
a first controller in electrical communication with the first heating element of the first vapor generating device and the first airflow generator, the first controller configured to vary voltage to one or more of the first heating element of the first vapor generating device and the first airflow generator;
a second vapor generating system comprising:
a second vapor generating device having a second heating element positioned within the second aperture, the second vapor generating device having a portion protruding through the carrier away from the base;
a second airflow generator positioned in the base and in fluid communication with the second vapor generating device; and
a second controller in electrical communication with the second heating element of the second vapor generating device and the second airflow generator, the second controller configured to vary voltage to one or more of the second heating element of the second vapor generating device and the second airflow generator; and
a container in fluid communication with the first and second vapor generating systems and configured to retain a quantity of vapor,
wherein the first controller is configured to cause the first vapor generating system to generate a quantity of a first vapor, and wherein the second controller is configured to cause the second vapor generating system to generate a quantity of a second vapor in a predetermined ratio to the quantity of the first vapor.

2. The vapor blending device of claim 1, further comprising a third vapor generating system comprising:
a third vapor generating device having a third heating element positioned within a third aperture of the carrier, the third vapor generating device having a portion protruding through the carrier away from the base;
a third airflow generator positioned in the base and in fluid communication with the third vapor generating device; and
a third controller in electrical communication with the third heating element of the third vapor generating device and the third airflow generator, the third controller configured to vary voltage to one or more of the third heating element of the third vapor generating device and the third airflow generator,
wherein the third controller is configured to cause the third vapor generating system to generate a quantity of a third vapor in a predetermined ratio to the quantity of the first and second vapors.

3. The vapor blending device of claim 2, further comprising a fourth vapor generating system comprising:
a fourth vapor generating device having a fourth heating element positioned within a fourth aperture of the carrier, the fourth vapor generating device having a portion protruding through the carrier away from the base;
a fourth airflow generator positioned in the base and in fluid communication with the fourth heating element of the fourth vapor generating device; and
a fourth controller in electrical communication with the fourth vapor generating device and the fourth airflow generator, the fourth controller configured to vary voltage to one or more of the fourth heating element of the fourth vapor generating device and the fourth airflow generator,
wherein the fourth controller is configured to cause the fourth vapor generating system to generate a quantity of a fourth vapor in a predetermined ratio to the quantity of the first, second, and third vapors.

4. The vapor blending device of claim 1, wherein the base further comprises an internal chamber at least partially surrounding the first and second vapor generating systems.

5. The vapor blending device of claim 1, further comprising a receptacle configured to sealingly couple with the container.

6. The vapor blending device of claim 5, further comprising a sealing plate positioned between the receptacle and the carrier, the sealing plate having a selectively sealing aperture configured to seal the vapor in the container when the receptacle is removed from the carrier.

7. The vapor blending device of claim 5, further comprising a sealing member positioned between the receptacle and the carrier, the sealing member configured to prevent vapor from entering the base.

8. The vapor blending device of claim 1, wherein the container is a flexible bag.

9. The vapor blending device of claim 1, wherein the first and second controllers are in communication with software using a wireless connection.

10. The vapor blending device of claim 9, wherein the software provides input to the first and second controllers to create the predetermined ratio of the quantity of the first and second vapors within the container.

11. A vapor blending device, comprising:
a carrier having a first aperture and a second aperture;
a first vapor generating device having a first heating element positioned within the first aperture and having a portion protruding through the carrier away from the base, the first vapor generating device in fluid communication with a first airflow generator positioned in the base and having a first controller in electrical communication with the first heating element of the first vapor generating device and the first airflow generator, the first controller configured to vary voltage to one or more of the first heating element of the first vapor generating device and the first airflow generator;
a second vapor generating device having a second heating element positioned within the second aperture and having a portion protruding through the carrier away from the base, the second vapor generating device in fluid communication with a second airflow generator positioned in the base and having a second controller in electrical communication with the second heating element of the second vapor generating device and the second airflow generator, the second controller configured to vary voltage to one or more of the second heating element of the second vapor generating device and the second airflow generator; and
a container in fluid communication with the first and second vapor generating devices and configured to retain a quantity of vapor,
wherein the first controller is configured to cause the first vapor generating system to generate a quantity of a first vapor, and wherein the second controller is configured to cause the second vapor generating system to generate a quantity of a second vapor.

12. The vapor blending device of claim 11, wherein the quantity of the second vapor is in a predetermined ratio to the quantity of the first vapor.

13. The vapor blending device of claim 12, further comprising a third vapor generating device having a third heating element positioned within a third aperture of the carrier and having a portion protruding through the carrier away from the base, the third vapor generating device in fluid communication with a third airflow generator positioned in the base and having a third controller in electrical communication with the third heating element of the third vapor generating device and the third airflow generator, the third controller configured to vary voltage to one or more of the third heating element of the third vapor generating device and the third airflow generator, wherein the third controller is configured to cause the third vapor generating system to generate a quantity of a third vapor in a predetermined ratio to the quantity of the first and second vapors.

14. The vapor blending device of claim 13, further comprising a fourth vapor generating device having a fourth heating element positioned within a fourth aperture of the carrier and having a portion protruding through the carrier away from the base, the fourth vapor generating device in fluid communication with a fourth airflow generator positioned in the base and having a fourth controller in electrical communication with the fourth heating element of the fourth vapor generating device and the fourth airflow generator, the fourth controller configured to vary voltage to one or more of the fourth heating element of the fourth vapor generating device and the fourth airflow generator, wherein the fourth controller is configured to cause the fourth vapor generating system to generate a quantity of a fourth vapor in a predetermined ratio to the quantity of the first, second, and third vapors.

15. The vapor blending device of claim 11, further comprising a base having an internal chamber at least partially surrounding the first and second airflow generators and the first and second controllers.

16. The vapor blending device of claim 11, further comprising a receptacle configured to sealingly couple with the container.

17. The vapor blending device of claim 16, further comprising a sealing plate positioned between the receptacle and the carrier, the sealing plate having a selectively sealing aperture configured to seal the vapor in the container when the receptacle is removed from the carrier.

18. The vapor blending device of claim 16, further comprising a sealing member positioned between the receptacle and the carrier, the sealing plate configured to prevent vapor from entering the base.

19. The vapor blending device of claim 11, wherein the container is a flexible bag.

20. The vapor blending device of claim 11, wherein the first and second controllers are in communication with software using a wireless connection, and wherein the software provides input to the first and second controllers to create the predetermined ratio of the quantity of the first and second vapors within the container.
